(19) United States
(12) Patent Application Publication
(10) Pub. No.: US 2012/0288959 A1
(43) Pub. Date: Nov. 15, 2012

(54) RAPID DETECTION OF CEREBROSPINAL FLUID, METHODS AND SYSTEMS THEREFORE

(75) Inventors: Wesley Palmer, Walnut Creek, CA (US); Michael Lee, Walnut Creek, CA (US); Richard Young, Alpine, UT (US); Munisch Gupta, Carmichael, CA (US)

(73) Assignee: ACROTECH SYSTEMS INC., Walnut Creek, CA (US)

(21) Appl. No.: 13/377,146

(22) PCT Filed: Jun. 8, 2010
(86) PCT No.: PCT/US2010/037854

§ 371 Date: Jul. 30, 2012

Related U.S. Application Data
(60) Provisional application No. 61/185,063, filed on Jun. 8, 2009.

(51) Int. Cl. G01N 33/566; G01N 21/78

(52) U.S. Cl. 436/501; 422/69

(57) ABSTRACT

Methods and systems are disclosed for rapid detection of cerebrospinal fluid (CSF) in a sample. In some embodiments, the methods comprise depleting a biological sample of beta-1 transferrin by contacting the sample with a sialic acid-specific lectin bound to a solid support, followed by subjecting the beta-1 transferrin-depleted sample to a lateral flow immunoassay. The methods can be used to detect CSF comprised by a sample in under one hour. Furthermore, the methods can detect CSF in a biological sample such as a plasma sample of a volume as small as about 10 μl.

### PRIORITY CLAIM

This application claims priority of U.S. Provisional Application Ser. No. 61/185,063, entitled “ELECTROCHEMICAL RAPID DETECTION OF CEREBROSPINAL FLUID, METHODS AND APPARATUSES THEREFORE,” filed on Jun. 8, 2009, which is incorporated herein by reference in its entirety.

### FIELD

Embodiments of the invention disclosed herein relate generally to methods for determining the presence, absence, or quantity of cerebral spinal fluid a biological sample.

### BACKGROUND

Cerebrospinal fluid (CSF) leakage is an escape of the fluid that surrounds the brain and spinal cord, from the cavities within the brain, or from the central canal in the spinal cord (1). CSF rhinorrhea or otorrhea occurs when there is a leakage of CSF from the subarachnoid space into the nasal or aural mucosa due to trauma, paranasal sinus disease, or surgery (1). If untreated, CSF leaks can cause life-threatening infections and brain abscesses. The risk of a meningitis infection is high, with reported rates varying between about 2-about 88% (2).

There are many disadvantages to current detection methods for CSF leakage. Currently patient samples must be packaged and shipped to laboratories, which can lead to lost, damaged or compromised samples. Highly trained technicians are required to perform the testing and results may take up to four days to obtain. IFE gels require large volumes of sample and concentration steps are usually performed prior to testing (7). A rapid, sensitive and cost-effective diagnostic test for cerebrospinal fluid can be a very useful tool in the medical industry.

### SUMMARY

A rapid, sensitive and cost-effective diagnostic test for detecting cerebrospinal fluid in a sample is needed in the medical and research industries. Accordingly, embodiments of the invention described herein relate to a method and an assay system for determining presence, absence or quantity of cerebral spinal fluid in a biological sample that includes lateral flow immunoassay technology. As described herein, the methods can be used to detect CSF comprised by a sample in under one hour and using relatively small volumes of biological sample.

Hence, in various aspects, embodiments herein relate to systems and methods for the rapid determination of the presence, absence or quantity of cerebral spinal fluid in a biological sample. In various configurations, these methods include: a) forming a beta-1 transferrin-treated sample by contacting a biological sample with lectin immobilized on a solid support, wherein the lectin is a sialic acid-specific lectin; and b) subjecting the beta-1 transferrin-treated sample to a lateral flow immunoassay. In some embodiments, a beta-1 transferrin-treated sample comprises a beta-1 transferrin-depleted sample, wherein the beta-1 transferrin-depleted sample is formed by separating beta-1 transferrin from the biological sample. Thus, in some embodiments, the beta-1 transferrin-treated sample is a beta-1 transferrin-depleted sample that is substantially free of beta-1 transferrin. In some embodiments, the sample is depleted of beta-1 transferrin prior to subjecting the sample to a lateral flow immunoassay. In other embodiments, the beta-1 transferrin treated sample comprises beta-1 transferrin that is specifically treated in way that facilitates separation of beta-1 transferrin from the sample and the beta-1 transferrin is separated following addition of the sample to a sample pad.

Some embodiments relate to systems for determining presence, absence or quantity of cerebral spinal fluid in a biological sample. In various configurations, these systems include a separation section to separate beta-1 transferrin from a biological sample to form a beta-1 transferrin-depleted sample. These systems include a lateral flow immunoassay section to qualitatively and/or quantitatively determine the presence of transferrin in the beta-1 transferrin-depleted sample. In some embodiments, the separation section and the lateral flow immunoassay section are integrated into an on-board system. In other embodiments, the separation section and the lateral flow immunoassay section are unconnected sections of an off-board system. For example, in some embodiments, the system comprises a kit comprising a separation section and a lateral flow immunoassay section.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates lateral flow assays for transferrin purified from human plasma.

FIG. 2 illustrates transferrin removal using SNA lectin as shown by lateral flow assays.

FIG. 3 illustrates sensitivity of detection of human cerebrospinal fluid using lateral flow assays.

FIG. 4 illustrates that SNA-1 gel is specific for beta-1 transferrin and does not react with beta-2 transferrin.

FIG. 5 illustrates sensitivity of lateral flow assays to transferrin levels in dilutions of normal human plasma samples.

FIG. 6 illustrates depletion of beta-1 transferrin using SNA gel.

FIG. 7 illustrates lateral flow assays comparing treatment of plasma with SNA gel to treatment of plasma/CSF with SNA gel.

### DETAILED DESCRIPTION OF THE INVENTION

Beta-1 transferrin is an iron-binding and transport protein found in human plasma, serum and other bodily fluids (3,4). Beta-2 transferrin is an isoform of transferrin that is structurally different from beta-1 transferrin and occurs in cerebrospinal fluid (CSF), perilymph and ocular fluids, but rarely in serum or plasma (5,6). Beta-2 transferrin is a useful tool in differentiating CSF from serum and plasma and is an important marker of CSF leakage.

The most common clinical method for detection of CSF leaks is Immunofixation Electrophoresis (IFE). This laboratory technique uses gel electrophoresis to detect beta-2 transferrin in human plasma and serum samples.

U.S. Pat. No. 5,702,904 to Makhlouf et al. discloses an antibody which reacts selectively with a transferrin homolog found in alcoholic but not in non-alcoholics. U.S. Pat. No. 6,737,278 to Carlsson et al. describes a method and device for determining an analyte by means of binding reactions. The method, while involving flow of an analyte on a flow matrix having a detection zone. U.S. Pat. No. 6,716,641 to Sundrehagen discloses a dipstick assay for detecting and quantifying the content of a target analyte in a sample for use in determining carbohydrate-free transferrin in a sample. U.S. Pat. No. 7,166,473 to Sundrehagen et al. discloses an assay method for assessing a transferrin varian for diagnosis and monitoring of alcoholism, including turbidimetric and nephelometric assays for determining transferrin content in a sample. US Patent Application Publication 2005/0239137 to Sundrehagen discloses a method for determination of carbohydrate-free transferrin in a body fluid for use in the assessment of alcohol consumption. However, none of these references describe an assay for the presence, absence or quantity of cerebral spinal fluid in a biological sample as described by the inventors herein.

Some embodiments of the invention relate to a system for the rapid determination of the presence, absence or quantity of cerebral spinal fluid in a biological sample. The system can separate beta-1 transferrin from a biological sample to form a beta-1 transferrin-depleted sample which can then be evaluated to detect the presence, absence or quantity of transferrin in the beta-1 transferrin-depleted sample using lateral flow immunoassay techniques.

In some embodiments, the system or device comprises a separation section and a lateral flow immunoassay section that are integrated into an on-board system. Merely by way of example, an on-board system refers to one whose separation and lateral flow immunoassay sections are portable and/or used as a single unit. In another example, an on-board system refers to one whose separation section and lateral flow immunoassay section are in fluid communication. In a further example, an on-board system refers to one in which the different sections of the system are in fluid communication, and therefore, the sample can advance from one section to another section without exposure to the ambient outside of the system or its cover; or without user's direct manipulation of the sample or its container within the system. In some embodiments, advance of the sample from one section to another within an on-board system can be initiated in a controlled fashion automatically depending on, for example, time, or semi-automatically by, for example, a user, or a combination thereof. The specific maneuver by a user depends on the mechanism of the flow control employed in an on-board system. Some exemplary manipulations by a user include, for example, but not limited to, removing a partition between adjacent sections, applying an external pressure to the cover of the system to push the sample over from one section to another, releasing or reducing a resistance force for the flow to advance, changing orientation of the system to manipulate the alignment of the flow and gravity, or the like, or a combination thereof. Merely by way of example, an on-board system includes a separation section and a lateral flow immunoassay section packaged within a cover. At a pre-determined time following application of a biological sample to the system (for example 5 minutes as pre-set by the manufacturer or user) during which the beta-1 transferrin is treated within the sample, the beta-1 transferrin-treated sample advances to the lateral flow immunoassay section with minimal or no user intervention.

In other embodiments, the separation and the lateral flow immunoassay sections are unconnected sections of an off-board system. Merely by way of example, an off-board system refers to a kit comprising a separation section and a lateral flow immunoassay section as separate units. In another example, an off-board system refers to one whose separation section and lateral flow immunoassay section are not in fluid communication. In a further example, an off-board system refers to one in which the different sections of the system are not in fluid communication, and therefore, the sample is transferred from one section to another section by a separate means including, for example, a pipette, a test tube, a container, a dish, or the like, which is not attached to the separation section and/or the lateral flow immunoassay section.

Merely by way of example, an off-board system comprises a separation section and a lateral flow immunoassay section which are not in fluid communication. After the sample is incubated in the separation section for a pre-determined amount of time sufficient to treat beta-1 transferrin within the sample, the sample is transferred to the lateral flow immunoassay section using a pipette by a user or a machine, wherein pipette is not attached to the separation section and/or the lateral flow immunoassay section, and therefore is discarded after use without disturbing the function and/or structure of the separation section and/or the lateral flow immunoassay section and/or the system.

Whether an on-board or off-board arrangement, in some embodiments, the beta-1 transferrin-treated sample is transferred from the separation section to the lateral flow immunoassay section without prior separation of the treated beta-1 transferrin, such as by filtration or other similar mechanical separation means, and the separation occurs in the lateral flow immunoassay section, such as at the sample pad. In some embodiments, the beta-1 transferrin-treated sample is subject to a separation step, such as filtration or other similar mechanical means, to form a beta-1 transferrin-depleted sample prior to transferring the sample to the lateral flow immunoassay section.

Thus, in some embodiments, beta-1 transferrin is removed from a sample, while leaving the desired beta-2 form behind. Without being limited by theory, it is believed that the beta-2 form of transferrin, also called asialo-transferrin, does not contain sialic acid residues and therefore will not bind to a sialic acid-specific lectin. Hence, a solid support having a sialic acid-specific lectin, such as Allomyrina dichotoma agglutinin (allo A) or Sambucus nigra lectin (SNA-I) can be used to deplete beta-1 transferrin from a biological sample.

Many different nitrocellulose membranes with different pore sizes and wicking rates can be used to separate the beta-1 transferrin-lectin conjugate from the remaining of the sample. Preferably, a membrane with a relatively slow wicking rate is used. A slow wicking rate can allow for increased time for the conjugate and sample to incubate as they migrate through the membrane, improving assay sensitivity. In a preferred embodiment, a HF240 membrane (Millipore) with a capture antibody concentration of about 1.0 mg/ml is used.

Conjugate chemistries, pH, protein levels, and concentrations can be optimized to produce better visibility of the test result signal, even flow across the entire test strip, and otherwise enhance assay performance. In some embodiments, about 6 μ/cm of conjugate at an optical density of about 10.0 provides an optimal dispense rate, producing intense pink/red signals at the test zone for positive samples, flowing evenly across the nitrocellulose membrane and releasing completely off the conjugate pad.

In preferred embodiments, Running Buffer comprising proteins, surfactants and polymers is used to enhance assay performance. Preferably, the Running Buffer creates stronger and brighter test line signals for positive samples, compared to a potassium phosphate buffer, with no signals for negative samples. Preferably, Running Buffer also improves the flow of fluids across the test strip. In a preferred embodiment, an even flow of conjugate on the membrane is achieved and clean membranes with no staining are observed at about fifteen minutes. In preferred embodiments, assay run time is about fifteen or twenty minutes.

In some embodiments, the separation section is for separating beta-1 transferrin from a biological sample to form the beta-1 transferrin-depleted sample. In some embodiments, the separation section is for specifically treating beta-1 transferrin within the biological sample to form the beta-1 transferrin-treated sample. The separation section includes a solid support having lectin.

In some embodiments, the separation section includes a lectin gel comprising beads coated with lectin suspended in a solution, wherein the beads function as the solid support. The suspension solution can comprise, for example, PBS. The suspension can further comprise a preservative. The solution or the preservative is not reactive with the biological sample, or at least not reactive with the analyte of interest (for example, beta-2 transferrin) of the sample.

The concentration of the beads in the suspension can be about 10%, or about 20%, or about 30%, or about 40%, or about 50%, or about 60%, or about 70%, or about 80% by volume. The concentration of the beads in the suspension can be at least about 10%, or at least about 20%, or at least about 30%, or at least about 40%, or at least about 50%, or at least about 60%, or at least about 70%, or at least about 80% by volume. The concentration of the beads in the suspension can be lower than about 80%, or lower than about 70%, or lower than about 60%, or lower than about 50%, or lower than about 40%, or lower than about 30%, or lower than about 20% by volume. The concentration of the beads in the suspension can be from about 10% to 20%, or from about 20% to about 30%, or from about 30% to about 40%, or from about 40% to about 50%, or from about 50% to about 60%, or from about 60% to about 70%, or from about 70% to about 80%, or above about 80% by volume. The concentration of the beads in the suspension can be from about 10% to 90%, or from about 20% to about 80%, or from about 30% to about 70%, or from about 40% to about 60%, or about 50% by volume. Merely by way of example, the beads comprise Acrobeads, Sepharose beads, magnetic beads, or the like, or a combination thereof.

The ratio of the biological sample to the lectin gel can be about 50:1, or about 40:1; or about 30:1; or about 25:1; or about 20:1, or about 18:1; or about 15:1; or about 12:1; or about 10:1; or about 9:1; or about 8:1; or about 7:1; or about 6:1; or about 5:1; or about 4:1; or about 3:1; or about 2:1; about 1:1, or about 1:2, or about 1:3, or about 1:4, or about 1:5, or about 1:6, or about 1:7, or about 1:8, or about 1:9, or about 1:9, or about 1:10; or about 1:12; or about 1:15; or about 1:18; or about 1:20; or about 1:25, or about 1:30; or about 1:40; or about 1:50; or about 1:60; or about 1:70; or about 1:80; or about 1:90; or about 1:100, or lower than about 1:100 by volume.

The beads are chemically and/or mechanically stable in presence of the biological sample and in the suspension solution.

In other embodiments, the separation section includes a sample pad comprising, for example, a nitrocellulose membrane with a specific pore size and wicking rate, wherein the membrane functions as the solid support and is coated with lectin. In some embodiments, the sample pad is located in the lateral flow immunoassay section. The separation section can further include beads, for example, Acrobeads, Sepharose beads, magnetic beads, or the like, or a combination thereof, wherein the beads can be retained within the membrane and cannot move to the lateral flow immunoassay section with the sample. The incubation to form the beta-1 transferrin-lectin conjugate can occur in a separate container, for example, in a tube or other similar container, before the sample is transferred to the sample pad comprising the membrane. The sample can be transferred using, for example, a pipette by a user. The incubation to form the beta-1 transferrin-lectin conjugate can occur in or on the sample pad comprising the membrane.

As used herein, the incubation time refers to the time when a biological sample is in direct contact with the lectin coated on the solid support. The incubation time can be at least 0.1 minutes, or at least about 0.5 minutes, or at least about 1 minute, or at least about 1.5 minutes, or at least about 2 minutes, or at least about 2.5 minutes, or at least about 3 minutes, or at least about 3.5 minutes, or at least about 4 minutes, or at least about 4 5 minutes, or at least about 5 minutes, or at least about 5.5 minutes, or at least about 6 minutes, or at least about 6.5 minutes, or at least about 7 minutes, or at least about 7 5 minutes, or at least about 8 minutes, or at least about 8.5 minutes, or at least about 9 minutes, or at least about 9.5 minutes, or at least about 10 minutes, or at least about 12 minutes, or at least about 15 minutes, or at least about 20 minutes, or at least about 25 minutes, or at least about 30 minutes. The incubation time can be less than 60 minutes, or less than about 50 minutes, or less than about 40 minutes, or less than about 30 minutes, or less than about 25 minutes, or less than about 20 minutes, or less than about 15 minutes, or less than about 12 minutes, or less than about 10 minutes, or less than about 8 minutes, or less than about 5 minutes, or less than about 3 minutes, or less than about 1 minute. The incubation time can be from about 0.1 minutes to about 60 minutes, or from about 0.5 minutes to about 50 minutes, or from about 1 minute to about 40 minutes, or from about 2 minutes to about 30 minutes, or from about 3 minutes to about 20 minutes, or from about 4 minutes to about 15 minutes, or from about 5 minutes to about 10 minutes.

After incubation, the biological sample can exit the separation section. The beads can be retained in the separation section by size, charge, magnetic force, or the like, or a combination thereof. Merely by way of example, the system comprises one or more filters at the exit of the separation section. The filter can comprise a filter paper, a filter tip, or the like, or a combination thereof. The size and/or shape of the pores in the filter can be chosen such that a majority of the beads and/or the beads with the beta-1 transferrin-lectin conjugate cannot pass through the pores. In another example, the system comprises a membrane without beads, wherein a majority of the beta-1 transferrin-lectin conjugate is retained within the membrane because of the size and/or shape or charges of the beta-1 transferrin-lectin conjugate which cannot pass through the membrane. In a further example, the system comprises a membrane with beads coated with lectin, wherein a majority of the beta-1 transferrin-lectin conjugate is retained within the membrane because of the size and/or shape or charges of the beta-1 transferrin-lectin conjugate binding on the beads which cannot pass through the membrane, or because of a magnetic retention force if the beads are magnetic beads. Merely by way of example, at least about 50%, or at least about 60%, or at least about 70%, or at least about 80%, or at least about 85%, or at least about 90%, or at least about 95%, or at least about 96%, or at least about 97%, or at least about 98%, or at least about 99% of the beads and/or and/or the beads with the beta-1 transferrin-lectin conjugate cannot pass through the filter. If the beads are magnetic, a majority of the beads and/or the beads with the beta-1 transferrin-lectin conjugate can be retained in the separation section using a magnetic force. Merely by way of example, at least about 50%, or at least about 60%, or at least about 70%, or at least about 80%, or at least about 85%, or at least about 90%, or at least about 95%, or at least about 96%, or at least about 97%, or at least about 98%, or at least about 99% of the beads and/or and/or the beads with the beta-1 transferrin-lectin conjugate can be retained in the separation section. At least about 50%, or at least about 60%, or at least about 70%, or at least about 80%, or at least about 85%, or at least about 90%, or at least about 95%, or at least about 96%, or at least about 97%, or at least about 98%, or at least about 99% of the beta-2 transferrin in the biological sample can be transferred to the lateral flow immunoassay section of the system.

Beta-1 transferrin in the biological sample can bind to the lectin-coated solid support. In this way, beta-1 transferrin in the biological sample can be retained in the separation section or at the sample pad, while beta-2 transferrin can move through the lateral flow immunoassay. Using the lectin gel, at least about 80%, or at least about 85%, or at least about 90%, or at least about 95%, or at least about 96%, or at least about 97%, or at least about 98%, or at least about 99%, or more than 99% of beta-1 transferrin in the sample is removed. The beta-1 transferrin-depleted sample can comprise beta-1 transferrin at non-detectable concentration. At least about 50%, or at least about 60%, or at least about 70%, or at least about 80%, or at least about 85%, or at least about 90%, or at least about 95%, or at least about 96%, or at least about 97%, or at least about 98%, or at least about 99%, or more than 99% of transferrin in the beta-1 transferrin-depleted sample is beta-2 transferrin. In some embodiments, in a beta-1 transferrin-depleted sample, beta-1 transferrin in the sample can be less than about 1% by weight of total protein in the sample, or less than about 0.1% by weight of total protein in the sample, or less than about 0.01% by weight of total protein in the sample, or less than about 0.001% by weight of total protein in the sample, or less than about 0.0001% by weight of total protein in the sample.

It is understood that the parameters and the exemplary values described above including size, and/or shape, and/or charge of the beads, the volume ratio of the beads to the suspension solution, the volume ratio of the biological sample to the lectin gel, the size or other properties of the membrane, or the like, are for illustration purposes only, and is not intended to limit the scope of the application. These parameters, alone or in combination, can be chosen to optimize the measurement. Merely by way of example, beads are used in the gel to increase the surface area for the binding of beta-1 transferrin with lectin to occur so that the chances for beta-1 transferrin to bind to lectin is increased; presence of the beads in the gel can also increase the incubation time; moreover, the beads can facilitate to retain bound beta-1 transferrin in the separation section by forming a beta-1 transferrin-lectin conjugate. These and other functions of the beads can depend on the parameters including, for example, size, shape, charge, concentration of the beads. Accordingly, these parameters, alone or in combination, can be chosen to optimize the operation parameters including the binding, the incubation time, the separation efficiency, or the like, or a combination thereof. It is understood that various operation parameters can have even or uneven weight in optimizing the overall performance of the measurement. Merely by way of example, if the separation efficiency is more important than the incubation time in determining the presence, absence or quantity of cerebral spinal fluid in a biological sample, the parameters of the system or a portion thereof are chosen to optimize the separation efficiency, if necessary, at the cost of less desirable incubation time.

It is understood that a gel with lectin coated beads is described herein for illustration purposes only, and is not intended to limit the scope of the application. Other types of solid support, for example, a porous matrix, which can provide desirable operation parameters, some of which are exemplified above, can be used. Merely by way of example, forming a beta-1 transferrin-treated sample or a beta-1 transferrin-depleted sample can comprise contacting the original biological sample with a sialic acid-binding lectin conjugated to a solid support such as, without limitation, a Sepharose® bead (GE Healthcare, Piscataway, N.J.). A sialic acid-specific lectin of these configurations can be, for example, Allomyrina dichotoma agglutinin (allo A) or Sambucus nigra lectin (SNA-I).

It is understood that lectin is described above in the embodiments as the binding partner or capture agent for beta-1 transferrin for illustration purposes only, and is not intended to limit the scope of the application. It is understood that other types of binding partners can be used as long as one can specifically bind to beta-1 transferrin but not beta-2 transferrin.

The beta-1 transferrin-treated sample or the beta-1 transferrin-depleted sample can advance or be transferred to the lateral flow immunoassay section as described above.

In some embodiments, when testing normal human plasma samples and pure human plasma a “high dose hook effect” can be observed. “High dose hook effect” is a common phenomenon in which a strong positive sample produces a negative test result. The high concentration of transferrin in human plasma samples can cause the “high dose hook effect.” The excess transferrin in the sample may not bind to all of the antibodies in the conjugate and can bind to the antibodies at the test line, preventing the conjugate complex from binding in the test zone. To prevent the “hook effect” from occurring, plasma samples can be diluted in a buffer prior to testing. In a preferred embodiment, a dilution factor of about 1 to 1600 is used to produce an optimal positive signal for beta-1 transferrin and no signal in the presence of SNA-gel.

When testing human CSF samples a detection limit of about 1 to 320 was determined when adding about 90 μl of diluted sample to the test device. Due to the high concentration of beta-1 transferrin in plasma, in a preferred embodiment, a sample volume of about 10 μl is used. When testing about 10 μl of diluted CSF the limit of detection for beta-2 transferrin was an about 1 to 20 dilution. Storage and handling conditions of the CSF prior to assay are preferably optimized for increased assay sensitivity in order to produce the most accurate assay result.

Lateral flow processes are well known. Lateral flow is a rapid immunoassay technology that has been widely used in the diagnostic industry since the 1980's. Typically, such diagnostics are performed as follows: a test sample is added to the test surface, typically followed by a chase buffer. The chase buffer allows for precise volumes of sample to be added to the test and facilitates the flow of fluids across the test surface. The sample and buffer re-hydrate dried conjugate, which contains a label substance, such as gold particles, that has antibodies attached. If the specific analyte is present in the sample it can bind to the labeled antibodies and the complex can migrate through the membrane by capillary action. The analyte and label complex can then bind to antibodies which are immobilized on the membrane, creating a visible indicator, such as a colored line, in the test zone. If no analyte is present in the sample, then the conjugate can migrate past the test zone and will not bind to the antibodies on the test line of the membrane. Optionally, a second line, called the control line, can capture and bind excess conjugate. In some embodiments, a control line is a procedural control, indicating the test was run properly. Typically, results can be read in about 5 to about 15 minutes. Except as otherwise noted herein, therefore, the process of embodiments of the invention described herein can be carried out in accordance with such known processes.

A lateral flow assay disclosed herein that can be easy to use and can accurately diagnose patients with a CSF leak. It can thus be used to save numerous lives, for example by providing warning of conditions for life-threatening infections by detecting CSF leaks in less than about 30 minutes, compared to four days using the current methods of detection. Time is of the essence when diagnosing life threatening ailments. A rapid immunoassay for CSF as described herein eliminates the need for skilled technicians to diagnose a CSF leak and allow doctors, paramedics and other professionals to run a test themselves. Rapid tests are relatively inexpensive and easy to use and can be used in a variety of settings, such as, for example, in an operating room for detection of CSF leaks during surgery, in an ambulance for detection of CSF leaks from trauma, or in the doctor's office for detection of CSF leaks due to paranasal sinus disease.

The principles and technology of lateral flow devices are well known, but specific aspects of the invention disclosed herein are set out in further detail below. In some embodiments, the lateral flow immunoassay includes: i) applying a beta-1 transferrin-treated sample or a beta-1 transferrin-depleted sample to a system configured for performing a lateral flow immunoassay, wherein the system comprises a test surface comprising two or more zones, for example, but not limited to, an application zone, a labeling zone, a detection zone, and optionally, a control zone, ii) flowing the sample on the test surface by capillary or wicking action, wherein the beta-1 transferrin-depleted sample contacts a labeled conjugate in a first zone, and wherein the transferrin, if any, forms a first complex comprising the transferrin and the labeled conjugate; iii) further flowing the beta-1 transferrin-depleted sample across the test surface by capillary or wicking action, wherein the first complex, if present, contacts a second conjugate in the second zone and forms a second complex comprising transferrin, the labeled conjugate, and the second conjugate.

Accordingly, one embodiment provides a lateral flow device for determining the presence, absence or quantity of cerebral spinal fluid in a beta-1 transferrin-depleted biological sample comprising: (a) an application zone for receiving a beta-1 transferrin-treated sample or a beta-1 transferrin-depleted sample; (b) optionally, a sample pad for separating treated beta-1 transferrin; (c) a labeling zone containing labeled binding partner for transferrin; and (d) a detection zone having an immobilized capture reagent for the transferrin. Thus, in some configurations, the device or apparatus can comprise a substantially horizontally disposed test surface comprising a first zone comprising a first conjugate comprising a first antibody directed against transferrin and a label, such as colloidal gold particles, and a second zone comprising a second antibody directed against transferrin, wherein the second antibody is immobilized on the test surface in the second zone.

The application zone in the device can be suitable for receiving beta-1 transferrin-treated or a beta-1 transferrin-depleted sample. It is typically formed from absorbent material such as blotting paper.

The labeling zone can contain a labeled conjugate which can bind to any transferrin in a beta-1 transferrin-depleted sample. For reasons of specificity, the conjugate can typically include an antibody. It will be appreciated that the term “antibody” may include polyclonal and monoclonal antibodies, as well as antibody fragments (e.g. F(ab)₂, Fc etc.), single chain Fvs etc., provided that the necessary binding activity and biological specificity are retained. The label can be one that allows for qualitative and/or quantitative detection. The label can be any substance that permits detection by the naked eye, such as, for example, but not limited to, colored latex beads, or silica, or liposomes that have encapsulated chemiluminescors (e.g., luciferin) or chromophores (e.g., dyes, or pigments). The label can also comprise a colloid system containing, for example, colloidal carbon or a dispersion of a metal such as gold or silver, which can be associated with the mobile binding member. Alternatively, the label can be a substance that is not particulate, such as, for example, a dye, a fluorophore, enzyme, or a chemiluminescor. For ease of detection, the label is preferably visible to the naked eye, for example, it is tagged with a fluorescent tag or, preferably, a colored tag such as conjugated colloidal gold, which is visible as a pink color. Accordingly, in a preferred embodiment, the labeling zone, or first zone, comprises a first conjugate comprising a first antibody directed against transferrin and colloidal gold particles.

The detection zone can retain transferrin to which label has bound. This can typically be achieved using an immobilized capture reagent, such as an antibody. Where the capture reagent and the label are both antibodies, they can recognize different epitopes on the hormone. This allows the formation of a “sandwich” comprising labeled conjugate-transferrin-immobilized capture reagent. In a preferred embodiment, the detection zone comprises a second antibody directed against transferrin, wherein the second antibody is immobilized on the test surface in the detection zone or second zone.

Preferably, the detection zone is downstream of the application zone, with the labeling zone typically located between the two. A sample can thus migrate from the application zone into the labeling zone, where any transferrin in the sample binds to the label. Transferrin-label complexes continue to migrate into the detection zone together with excess label. When the transferrin-label complex encounters the capture reagent, the complex is retained while the sample and excess label continue to migrate. As transferrin levels in the sample increase, the amount of label (in the form of transferrin-label complex) retained in the detection zone increases proportionally.

In some embodiments of the invention, the system includes a control zone downstream of the detection zone. This can generally be used to capture excess label which passes through the previous zones (for example, using immobilized anti-label antibody). Thus, in some embodiments, the lateral flow immunoassay section further comprises a third zone comprising a third conjugate directed against the first conjugate, wherein the third conjugate is immobilized on the test surface in a third zone. In these embodiments, the immunoassay further comprises flowing the beta-1 transferrin-depleted sample to the third zone. In these configurations, the first conjugate, if not already immobilized by the second conjugate in the second zone, forms a complex with a third conjugate. For example and without limitation, if the first antibody is a polyclonal rabbit antibody directed against transferrin, the third antibody can be a goat anti-rabbit antibody. The appearance of color in the third zone can thus indicate that the conjugate comprising colloidal gold and the first antibody is flowing by capillary action during an assay. When label is retained at the control zone, this confirms that mobilization of the label and migration through the device have both occurred. It will be appreciated that this function may be achieved by the reference zone.

Furthermore, in some configurations, the color signal can also be assessed qualitatively and/or quantitatively by methods well known to skilled artisans, such as those presented elsewhere herein. Thus, in some embodiments, the methods described herein can further comprise determining presence, absence or quantity of the second complex in the detection or second zone. For example, in one embodiment, when the second complex comprises transferrin and colloidal gold particles, a red color visible to the unaided eye appears. The intensity of the color can be monotonically related to the amount of transferrin in the second complex. Because under the test conditions, the amount of transferrin present is monotonically related to the amount of cerebrospinal fluid in the sample, the intensity of the color provides an indication of the presence, absence, and/or quantity of cerebrospinal fluid comprised by the sample. In some configurations, the intensity of the color can be quantified by methods well known to skilled artisans, such as, without limitation, estimation by an unaided observer, measurement of light absorbance in a spectrophotometer, or quantification of pixels in a photograph of the test surface. In addition, the presence (or not) of accumulated red color in the second zone can provide a qualitative indication of the presence of cerebrospinal fluid in the biological sample. The methods of these configurations, therefore provide a qualitative and/or quantitative indication of the presence of cerebrospinal fluid in the sample.

The various zones are preferably formed on nitrocellulose.

Migration from the application zone to the detection zone can generally be assisted by a wick downstream of the detection zone to aid capillary movement. This wick is typically formed from absorbent material such as blotting or chromatography paper.

In some embodiments, the system is produced simply and cheaply. Merely by way of example, the system is conveniently in the form of a dipstick. Furthermore, it can be used very easily, for instance by a lay user. Embodiments of the invention thus provide a system which can be used as a screen for cerebrospinal fluid leakage.

In some embodiments, a biological sample which can be assayed for the presence of cerebrospinal fluid can comprise any tissue or body fluid, such as, for example and without limitation, an extract of a biopsy sample, a blood sample, a plasma sample, a serum sample, a nasal fluid sample, an aural fluid sample, or a lymphatic fluid sample, and the like. Before a biological sample enters the lectin gel, the sample can be pre-treated in order to remove, for example, cell debris, white and/or red blood cells, or the like, or a combination thereof.

In some embodiments, the immunoassay disclosed herein can detect cerebrospinal fluid in small samples. For example and without limitation, the volume of a biological sample or beta-1 transferrin-treated sample or a beta-1 transferrin-depleted sample that can be used in the methods can be less than about 100 μl, less than about 50 μl, less than about 20 μl, less than about 15 μl, or less than about 10 μl. Furthermore, in various configurations, the volume of sample can be from about 1 μl up to about 100 μl, from about 2 μl up to about 50 μl, from about 3 μl up to about 20 μl, or from about 5 μl up to about 10 μl.

In some embodiments, the methods can further comprise, optionally, diluting the sample with a buffer, prior to the applying to a lateral flow immunoassay section, at a volume ratio of sample: buffer of about 1:1, or about 1:2, or about 1:3, or about 1:4, or about 1:5, or about 1:6, or about 1:7, or about 1:8, or about 1:9, or about 1:10, or about 1:20, or about 1:50, or about 1:100, or about 1:160; or about 1:320; or about 1:400; or about 1:500, or about 1:800; or about 1:1000, or about 1:2500; or about 1:5000; or up to about 1:10,000.

In some embodiments, the first and second conjugates each are an anti-transferrin antibody. Each antibody can be any type of antibody known to skilled artisans, such as, and without limitation, a polyclonal antibody, a monoclonal antibody, or a transferrin-binding fragment of an antibody such as a Fab fragment. Such antibodies can be prepared by standard methods well known to skilled artisans such as methods set forth in Harlow, E., Using Antibodies: A Laboratory Manual, Cold Spring Harbor Laboratory Press, Cold Spring Harbor, N.Y., 1999, or can be purchased from a commercial supplier. In some configurations, the antibodies can be of different types, e.g., the first antibody can be a mouse monoclonal antibody and the second antibody can be a rabbit polyclonal antibody, or vice versa.

In some embodiments, the test surface comprises a material known to skilled artisans as effective for movement by capillary or wicking action of an aqueous solution, such as, without limitation, nitrocellulose membrane or paper. In some configurations, a test surface can be affixed to a support such as a backing card. In addition, in some configurations, the system further comprises an absorbent pad, such as a cellulose fiber pad. This pad can be used to introduce a sample to a test surface.

In various configurations of the present teachings, an assay can detect wherein the assay detects transferrin at a concentration as low as about 0.001 mg/ml.

Some embodiments of the invention also provide a process for measuring cerebral spinal fluid, comprising the steps of: (a) obtaining a biological sample; (b) forming a beta-1 transferrin-depleted sample by contacting a biological sample with lectin immobilized on a solid support, wherein the lectin is a sialic acid-specific lectin; (c) contacting the beta-1 transferrin-depleted sample with a label which binds to any transferrin in the sample; (d) separating transferrin-bound label; (e) detecting a signal associated with the separated label from step (d); and (f) comparing the signal detected in step (e) with a reference signal which corresponds to the signal given by a sample from a patient with a transferrin level equal to a threshold concentration.

In some embodiments, the time interval between the subjecting the biological sample to the solid support (for example, beads coated with lectin suspended in a solution, or porous matrix with lectin) and the appearance of a color signal in the second and/or third zone can be less than about 1 hr, less than about 30 minutes, less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, less than about 11 minutes, less than about 10 minutes, or less than 5 minutes. In some embodiments, the time interval can be about 1 minute.

Other objects and features will be in part apparent and in part pointed out hereinafter.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Removal of Beta-1 Transferrin from a Sample

In these experiments, tests were run to remove beta-1 transferrin using allo A lectin conjugated to Sepharose® beads.

### Example 2

### Test Surface Membranes

In this example, several nitrocellulose membranes with varying pore sizes and wicking rates were tested: Millipore: Catalog HF 135, HF 180, and HF 240; Whatman: Catalog FF 8511 00; MDI: Catalog 150 CNPH and 200 CNPH. A polyclonal antibody, affinity purified Goat anti-Human Transferrin, catalog number G-152-C, was purchased from BiosPacific. This antibody was used for immobilization onto the nitrocellulose membranes for the test line. The antibody was striped on the membranes at various concentrations: about 0.25 mg/ml, about 0.5 mg/ml, about 0.75 mg/ml, and about 1.0 mg/ml. The test line antibody was diluted in an about 25 mM potassium phosphate buffer (pH about 7.4), and dispensed onto the nitrocellulose at a rate of about 1.0 μl/cm. For the control line, Goat anti-Mouse at a concentration of about 0.5 mg/ml was used. The control line reagent was diluted in about 25 mM potassium phosphate (pH about 7.4) and dispensed onto the nitrocellulose at a rate of about 1.0 μl/cm. After striping, the membranes were dried on a heat plate for approximately two minutes. They were then stored in a desiccated container at room temperature overnight. Membranes were blocked using Millenia Diagnostics' Lateral Flow Blocking Buffer code: LBS, containing about 50 mM potassium phosphate, about 0.2% casein, about 0.5% PVA, about 0.1% surfactant 10G, and about 0.2% sucrose. Membranes were blocked by applying about 3 ml of block solution to the bottom of each membrane, allowed to wick up, incubated for two minutes, and dried on a heat plate for thirty minutes. The membranes were then stored in a desiccated container for further manufacturing use.

### Example 3

### Assay Optimization

For a conjugate pad, glass fiber was obtained from Ahlstrom, Cat. #8964 Glass, and treated with Millenia Diagnostics' Lateral Flow Blocking Buffer code: LBS. Glass fiber strips about 8 mm by about 30 cm were soaked in blocking solution (about 3 ml/strip) and incubated for about 5 minutes at room temperature. The glass fiber pads were then removed from the solution and placed on a heat plate lined with absorbent paper, to dry for about 1 hour. The pads were then placed in a desiccated container until used.

A monoclonal antibody to human transferrin, clone number A58210036P, was purchased from BiosPacific for conjugation and detection. The antibody was dialyzed in about 10 mM potassium phosphate (pH about 7.4) at about 1 mg/ml and conjugated to colloidal gold (40 nm particles) at a variety of protein and pH levels. Initially a protein titration was conducted to determine the optimal protein level for conjugation. Two milliliters of colloidal gold at pH about 9.0 was placed in several glass tubes. The antibody was then added in about 2.5 microliter increments, increasing from about 0 microliter to about 25 microliter, to each tube and conjugated to the gold particles. Unstable conjugates are observed by a color change from red to purple upon incubation with about 10% sodium chloride. For conjugates the tube containing about 12.5 μg/ml of antibody remained red. About 12.5 μg/ml of antibody was used for final conjugate preparations to ensure sufficient protein coating of the gold particles.

The gold was adjusted to various pH units using about 100 mM potassium carbonate. About fifty milliliters of colloidal gold was placed into a clean glass beaker. Potassium carbonate was added to adjust the pH in about 0.2 unit increments. About two milliliters of gold was removed when the gold solution reached pH about 7.4, about 7.6, about 7.8, about 8.0, about 8.2, about 8.4, and about 8.6. The antibody was then conjugated to the gold particles at each pH. Liquid testing of the conjugates was first performed to determine the optimal pH. The conjugates were prepared having an OD₅₂₀ nm of about 10.0. Conjugate at pH level about 8.6 was chosen for future scale-up and dry-down experiments. The conjugate was chosen based on visual inspections of the pellet after centrifugation, conjugate flow, signal intensity at the test line for positive samples, and no background signals for negative samples. Prior to dispensing the conjugates onto treated glass fiber pads, rewetting agents were added in the form of about 20% sucrose and about 5% trehalose (w/v). Each conjugate was dispensed onto the treated glass fiber at varying rates from about 4 μ1/cm to about 8 μl/cm, and dried at about 37° C. in a convection drying oven for about one hour. The conjugate pads were then stored in a desiccant cabinet until further use.

### Example 4

### Preparation of Samples

A variety of samples were used during this study: lyophilized Pure Human Transferrin from Athens Research and Technology, human plasma and serum samples provided by Millenia and a CSF sample from Javelin. The samples were diluted in two sample buffers: about 25 mM potassium phosphate buffer, and Running Buffer containing about 10 mM sodium phosphate, about 150 mM sodium chloride, about 3% BSA, about 15 mM EDTA, about 5% isopropanol, about 0.25% TWEEN-20, and about 0.095% sodium azide. Sambucus nigra lectin conjugated to sepharose beads from EY Laboratories, Inc. (San Mateo, Calif.) catalog number A-6802-2, was used throughout the study for removal of beta-1 transferrin from the sample. Various amounts of lectin, sample, and incubation times were used to determine the optimum conditions for beta-1 transferrin removal.

### Example 5

### Assay Assembly

Cards were assembled using backing cards obtained from G&L Precision Die Cutting, Inc. (San Jose, Calif.). A composite of paper and glass fiber from Ahlstrom (Helsinki, Finland), Grade 1660, was used as the sample application pad. Cellulose fiber from Ahlstrom, Grade 222, was used as the absorbent pad. The cards were assembled by removing the center adhesive cover from the backing card, and applying the membrane to the card. The top adhesive cover was removed and the absorbent pad applied with a 2 mm overlap onto the membrane. The bottom adhesive cover was then removed and the conjugate pad placed down with an about 2 mm overlap onto the membrane. The sample pad was then applied below the conjugate pad, aligning it with the bottom of the card. The cards were placed in a guillotine cutter and cut into test strips about 4.5 millimeters in width. The cut strips were placed into cassettes, and stored desiccated until tested.

### Example 6

### Assay Procedure

Positive and negative samples were applied to the sample pad at various volumes. It was determined that about 10 μl per test produced optimal results. Subsequently, about 10 μl of sample was added to the sample port of the cassette followed by about 80 μl of Running Buffer and a timer was started with an about 15-minute countdown. As the assays were run they were closely watched to monitor flow, conjugate release and the appearance of signals at the test and control regions. After about fifteen minutes the results were read and visual interpretations recorded.

### Example 7

### Assay Performance

A set of experiments was conducted to develop an antibody-gold conjugate. During the protein titration, sodium chloride (about 10%) was added and a color change of red to purple was noted for the tubes with lower levels of protein. This color change indicates there is not enough protein to stabilize the gold particles. About 125 μl of monoclonal anti-transferrin antibody (clone number A582 10036P) per about 10 ml of colloidal gold was used for conjugation and was necessary to stabilize the gold. Testing of the conjugates in liquid form at various pH levels was conducted by adding about 4 μl of each conjugate to a strip, about 10 μl of normal human plasma, followed by Running Buffer. Observations were made to monitor conjugate release, flow and signal intensity and based on these observations pH about 8.6 were chosen for scale-up and future testing.

A conjugate at pH about 8.6 and optical density of about 10.0 was scaled up and produced for dry-down testing. The conjugate was dispensed and dried onto treated glass fiber at varying concentrations: about 4, about 6 and about 8 μl/cm. Subsequently test strips were produced to evaluate the use of a dry-down conjugate and to test membranes of different pore sizes. Test strips were assembled using membranes from various vendors with a test line striped at about 1.0 mg/ml and conjugate dried down at about 6 μ1/cm. Human serum, a source for beta-1 transferrin, was tested at different volumes ranging from about 10 to about 40 μl. Running Buffer, provided by Millenia Diagnostics, was used as a chase and for a negative control. All test strips produced test line signals for human serum samples, indicating the test is likely detecting beta-1 transferrin, with varying degrees of signal intensity. Some membranes produced background signals at the test line when testing buffer and were eliminated from future testing. Test strips with membrane HF 240 produced the best results with the strongest test line signals when testing serum and clean membranes with no background when testing buffer. Membrane HF 180 produced similar results as HF 240 with slightly weaker test line signals and both membranes were chosen for future experiments.

A third set of experiments was conducted to determine optimal striping concentrations for the test line antibody. In this experiment human CSF, provided by Javelin Diagnostics, was evaluated. Membranes HF 240 and HF 180 from Millipore were tested with three test line concentrations: about 0.75 mg/ml, about 1.0 mg/ml and about 1.5 mg/ml. Human serum and CSF was added to the test strips at volumes ranging from about 1 μl to about 80 μl. Running Buffer was used as the chase buffer and for the negative control. All of the test strips produced test line signals for human serum and for CSF samples. This was an indication the test was detecting transferrin, beta-1 in human serum and both beta-1 and likely beta-2 in CSF. It was noted that test strips with membrane HF 240 and a test line concentration of about 1.0 mg/ml produced optimal results, creating the strongest test line signals for CSF and serum samples. Optimal assay run time was found to be about 15 minutes, allowing for a complete clearing of the conjugate across the membrane.

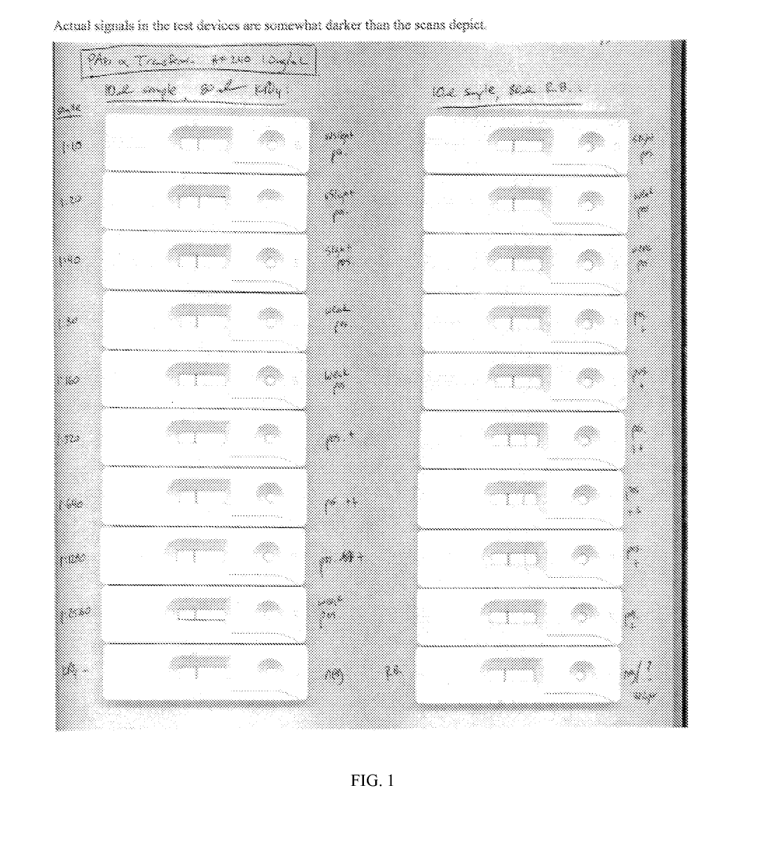

The next set of experiments was completed using purified transferrin from human plasma (lyophilized) obtained from Athens Research. This material was used to determine the analytical sensitivity of the assay for beta-1 transferrin. Purified transferrin is obtained from healthy human donors and should contain only the beta-1 form of transferrin. The purified transferrin was re-hydrated with about 25 mM potassium phosphate buffer pH about 7.4 to a concentration of about 10 mg/ml. The re-hydrated sample was serially diluted in a potassium phosphate buffer from about 1 to 10 (about 1.0 mg/ml) down to a dilution of about 1 to 2,560 (about 0.004 mg/ml). Test strips were assembled into cassettes prior to testing. Two chase buffers were evaluated in this experiment: about 25 mM potassium phosphate buffer and Running Buffer. Upon testing it was determined the assay had a “high dose hook effect,” where stronger samples produced very weak test line signals and as samples were diluted the test line signals increased. Purified transferrin at a dilution of about 1 to 640 (about 0.016 mg/ml) produced the strongest test line signals. Weak test line signals were observed for the lowest dilution of about 1 to 2,560 for both buffers tested. Running buffer, when used as a chase buffer, produced stronger and more visible test line signals than potassium phosphate buffer. These results can be observed in FIG. 1 (scans were taken during the report and not at the time of testing).

### Example 8

### Detection Sensitivity for Purified Transferrin

In these experiments for determining sensitivity of the detection using purified transferrin, serial dilutions were prepared from the about 1 to 2,560 dilution down to an about 1 to 20,480 dilution. Samples were tested using Running Buffer as chase. A slight signal was observed for the about 1 to 10,240 dilution and no signals were observed beyond that dilution. The limit of detection for pure human transferrin was established at an about 1 to 10,240 dilution (about 0.001 mg/ml).

Another experiment was set up to test the use of the monoclonal anti-transferrin antibody (clone number A58210036P) as the capture antibody at the test line and as the detector antibody in the conjugate. These test devices did not produce a visible test line for any of the diluted transferrin samples. This indicated the test needs the polyclonal antibody to be used for capture on the membrane with the monoclonal antibody in the conjugate for detection.

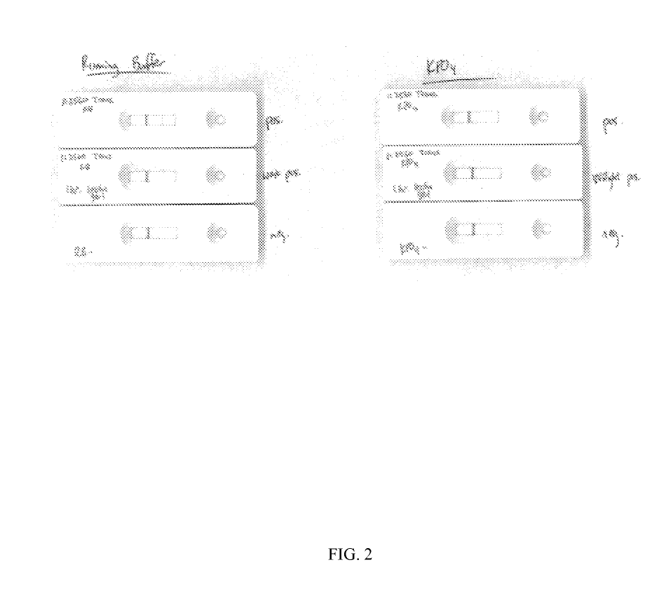

Samucus nigra (SNA) lectin conjugated to sepharose beads was obtained from EY laboratories for removal of beta-1 transferrin. Initial experiments were completed by incubating transferrin samples in a small plastic column with the lectin beads. About one milliliter of SNA beads (in phosphate buffer) was added to the column, followed by about 0.5 ml of pure transferrin diluted about 1 to 2,560. The sample and lectin incubated in the column at room temperature for about 1 hr. and at about 2 to about 8° C. for about 24 hours. The sample was collected from the column and tested (about 10 μl) with both potassium phosphate and Running Buffer (about 80 μl) as chase. A significant decrease in signal intensity was observed after one hour incubation in the column. Test line signals were almost eliminated after incubation with the gel, indicating beta-1 transferrin is being removed at some level. Incubation with the SNA-gel overnight had no observable effect on decreasing the test line signal compared to the one hour incubation, suggesting the reaction between the lectin and transferrin occurs within one hour. Results of purified transferrin (diluted to about 1 to 2560) incubated with the SNA-gel for one hour can be found in FIG. 2.

### Example 9

### Detection of Cerebral Spinal Fluid (CSF)

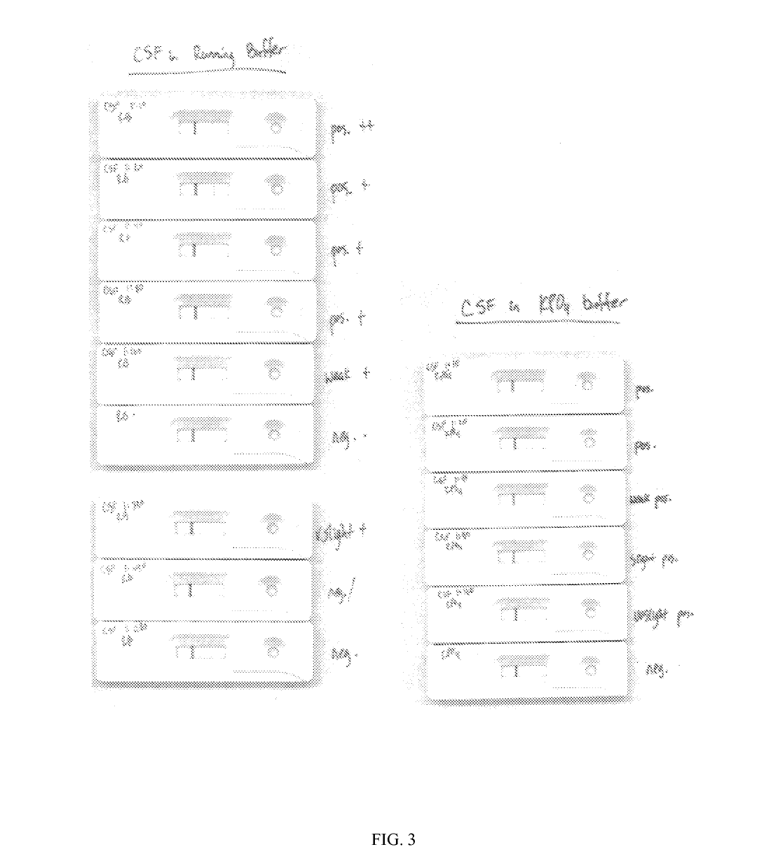

In these experiments, CSF was serially diluted in two buffers: potassium phosphate and Running Buffer. Serial dilutions of CSF were prepared starting with an about 1 to 10 dilution, down to an about 1 to 1,280 dilution. Initial experiments were conducted by adding about 10 μl of sample followed by about 80 μl of Running Buffer. Test line signals observed were weak for the about 1 to 10 dilution and no test line signals were observed past the about 1 to 20 dilution. Subsequent experiments were conducted by adding about 90 μl of the diluted CSF sample to the sample port of the test device and reading results at about 15 minutes. CSF diluted in potassium phosphate was detected at a dilution of about 1 to 160, while CSF in Running Buffer was detected down to a dilution of about 1 to 320. Running buffer created stronger test line signals than other buffers tested and increased the limit of detection of CSF by two fold. Scans of testing diluted CSF samples and comparing two chase buffers, can be found in FIG. 3.

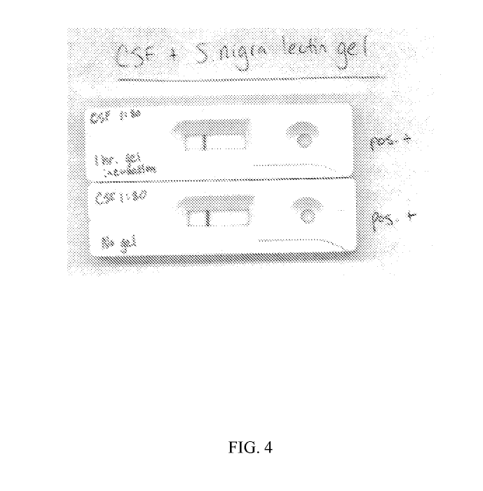

Once it was established that CSF was being detected in the rapid test, an experiment was designed to run a diluted CSF sample over the SNA-gel column A volume of about 1 ml of SNA beads (in phosphate buffer) was added to the column, followed by about 0.5 ml of diluted CSF sample. A weak positive dilution of CSF at about 1 to 80 was chosen to run over the SNA-gel column. The sample and gel incubated for one hour at room temperature, the sample was collected and subsequently tested. A sample of CSF diluted about 1 to 80 that was not run over the column was tested in parallel. A sample volume of about 90 μl was added to the test device and results were observed at about fifteen minutes. Positive test line signals were observed for both samples, with no visual difference in test line intensity for the CSF samples run over the gel and the sample not added to the column This indicated the SNA-gel is specific for beta-1 transferrin and doesn't appear to be reacting or binding to the beta-2 transferrin in the CSF sample. Results of this testing are displayed in FIG. 4.

### Example 10

### Detection of Beta-1 Transferrin in Human Plasma Samples

In these experiments, normal human plasma samples in sodium heparin were used for testing. These samples were obtained from healthy living donors and were expected to contain only the beta-1 form of transferrin. Three random plasma samples were thawed and tested in the assay. All three samples produced positive results with varying test line signal intensities. Plasma samples were then run over an SNA-gel column for removal of beta-1 transferrin. A variety of sample and gel volumes were tested with incubation times of fifteen minutes to about one hour. All initial attempts for beta-1 removal from human plasma samples were unsuccessful and positive test line signals were observed in every experiment. No visual difference was observed when adding plasma samples to the column compared to no incubation with the SNA-gel.

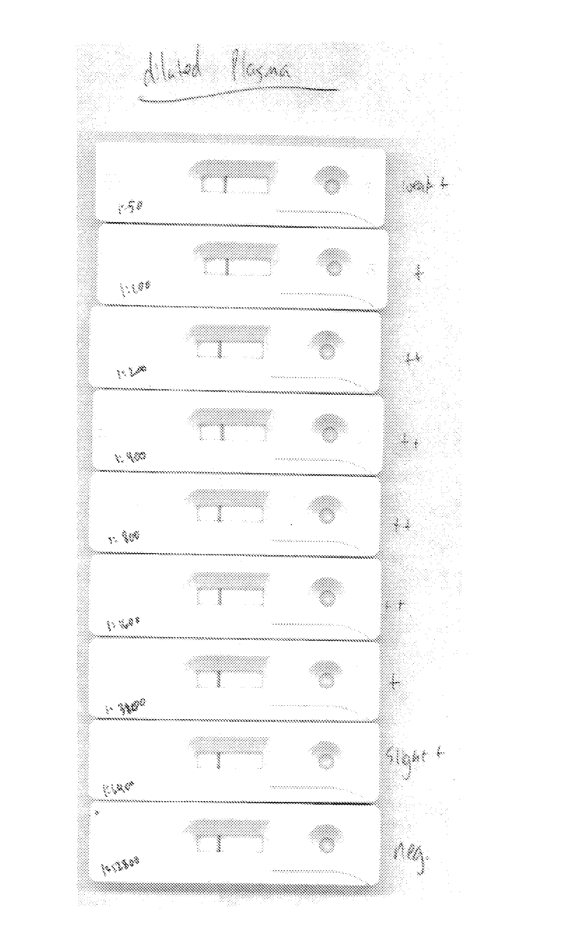

To determine the true sensitivity of the assay to transferrin levels in normal human plasma samples, a serial dilution of human plasma in about 25 mM potassium phosphate buffer (pH about 7.4) was performed and tested. As shown in FIG. 5, the results of this testing indicated a “high dose hook effect” of beta-1 transferrin in human plasma was occurring when testing normal human plasma.

### Example 11

### Sample Dilution

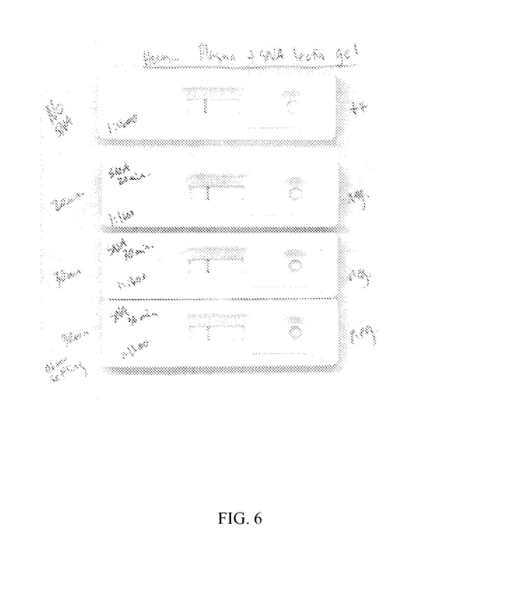

In these experiments, normal human plasma samples with the SNA-gel was diluted prior to incubation. A plasma sample dilution of about 1 to 1600 was chosen for testing because it produced a strong visual test line signal. Initially the diluted plasma was run over a column and tested. A decrease in signal intensity after one hour incubation with the lectin-gel was observed, indicating the beta-1 transferrin in the plasma was being removed at some level. In an attempt to have more successful binding of lectin and transferrin the plasma was added to an about 2 ml eppendorf tube containing about 0.5 ml of the SNA-gel (w/ buffer) and mixed. The use of a tube in place of the column would allow for constant mixing of the sample and gel, in hopes of having more effective removal of beta-1 transferrin. The plasma was added directly to the eppendorf tube at a dilution factor of about 1 to 1600 and thoroughly mixed. After an incubation time of about 20 min, about 30 min, and one hour the samples were tested A volume of about 10 μl of sample followed by about 80 μl of Running Buffer was added to the test devices. All samples tested negative at about 20, about 30 and about 60 min. This was an indication that beta-1 transferrin was successfully removed from the diluted plasma sample within about 20 minutes. The test mounts (FIG. 6) show diluted plasma with no gel incubation and diluted plasma at about 20 and about 30 min. gel incubations in an eppendorf tube. The results illustrate the importance of diluting a plasma sample prior to incubation.

### Example 12

### Detection in Simulated Plasma Samples Contaminated with CSF

In these experiments, normal human plasma was diluted in potassium phosphate buffer at two levels: about 1 to 400 and about 1 to 800. A diluted plasma sample of about 0.5 ml was added to an eppendorf tube containing about 0.5 ml of SNA-gel (with buffer). The diluted sample and lectin was mixed, incubated at room temperature and tested at time intervals of about 0, about 5, about 10, about 15 and about 20 minutes.

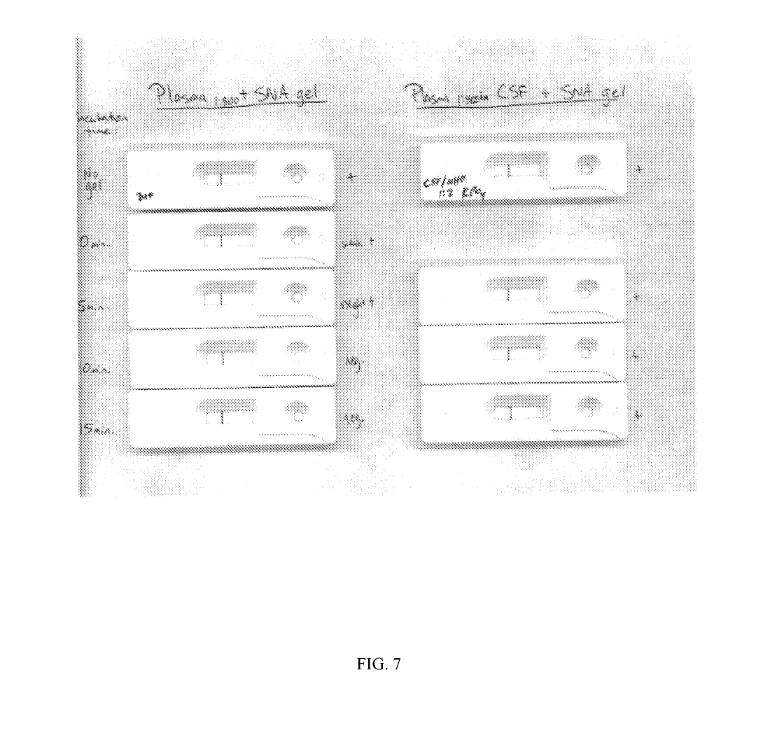

Samples were added to the test device (about 10 μl) followed by Running Buffer (about 80 μl) and results were read at fifteen minutes. Plasma diluted at about 1 to 400 produced a decrease in test line signals as the incubation time increased, but a slight signal was still observed at about 20 minutes. Plasma diluted at about 1 to 800 produced decreased test line signals at about 0 and about 5 minutes of incubation and no visible test line signals were observed at about 10, about 15 and about 20 minutes incubation time. A similar experiment was conducted diluting human plasma into CSF, representing a CSF leak and contamination of the plasma. Plasma was diluted about 1 to 800 in CSF and about 0.5 ml was added to an eppendorf tube containing about 0.5 ml of SNA-gel. After mixing and incubation periods of about 5, about 10 and about 15 minutes the sample was added to the test device. Test line signals were observed for each test device with no visible decrease in signal intensity as the incubation times increased. Results of comparing plasma with SNA gel and plasma/CSF with SNA gel can be observed in FIG. 7, and illustrates that a signal can be obtained in as little as 5 min, at a dilution of about 1:800.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about” or “substantially.” For example, “about” or “substantially” can indicate ±20% variation of the value it descries, unless otherwise stated. Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Fransen, P., C. J. M. Sindic, C. Thauvoy, C. Laterre, G. Stroobandt. 1991. Highly sensitive detection of beta-2 transferrin in rhinorrhea and otorrhea as a marker for cerebrospinal fluid leakage. Acta Neurochir. 109:98-101.
2. Brodie H A, Thompson T C. Management of complications from 820 temporal bone fractures. Am J Otol 1997: 18: 188-197.
3. Kjeldsberg CR, Knight JA. Body fluids: laboratory examination of amniotic, cerebrospinal, seminal, serous, and synovial fluids, 3rd ed 1993: 89-97 ASCP Press Chicago, Ill.
4. Verheecke P. On the τ-protein in cerebrospinal fluid. J Neurol Sci 1975; 26: 277-281.
5. Delaroche O, Bordure P, Lippert E, Sagniez M. Perilymph detection by 132-transferrin immunoblotting assay. Application to the diagnosis of perilymphatic fistulae. Clin Chim Acta 1996; 245: 93-104.
6. Tripathi R C, Millard C B, Tripathi B J, Noronha A. Tau fraction of transferrin is present in human aqueous humor is not unique to cerebrospinal fluid. Exp Eye Res 1990; 50: 541-547.
7. Irjala K, Suonpaa J, Laurent B. Identification of CSF leakage by immunofixation. Arch Otolaryngol 1979; 105: 447-448.

1. A method for determining presence, absence or quantity of cerebral spinal fluid in a biological sample, the method comprising: a) forming a beta-1 transferrin-depleted sample by contacting a biological sample with lectin coated on a solid support for a time, wherein the lectin is a sialic acid-specific lectin; b) subjecting the beta-1 transferrin-depleted sample to a lateral flow immunoassay, wherein the immunoassay comprises: i) applying the transferrin-depleted sample to a lateral flow immunoassay section, wherein the section comprises a test surface comprising a first zone comprising a first conjugate comprising a first antibody directed against transferrin and colloidal gold particles, and a second zone comprising a second antibody directed against transferrin, wherein the second antibody is immobilized on the test surface in the second zone; ii) flowing the beta-1 transferrin-depleted sample on the test surface by capillary action, wherein the beta-1 transferrin-depleted sample contacts the first conjugate in the first zone, and wherein the transferrin, if any, comprised by the beta-1 transferrin-depleted sample forms a first complex comprising the transferrin comprised by the beta-1 transferrin-depleted sample, and the first conjugate; iii) further flowing the beta-1 transferrin-depleted sample across a second zone, wherein the first complex, if present, contacts a second antibody in the second zone and forms a second complex, wherein the second complex comprises the transferrin comprised by the beta-1 transferrin-depleted sample, the first antibody, the colloidal gold, and the second antibody.

2. The method of claim 1, wherein the solid support comprises a bead.

3. The method of claim 2, wherein the bead comprises at least one selected from an Acrobead, a Sepharose® bead, and a magnetic bead.

4. The method of claim 1, wherein the time is from about 1 to about 20 minutes.

5. (canceled)

6. The method of claim 1, wherein the test surface further comprises a third zone comprising a third antibody directed against the first antibody, wherein the third antibody is immobilized on the test surface in the third zone, and wherein the immunoassay further comprises further flowing the beta-1 transferrin-depleted sample to the third zone, and wherein the first conjugate, if any, forms a third complex with the third antibody.

7. The method of claim 1, further comprising determining presence, absence or quantity of the second complex comprised by the second zone.

8. The method of claim 7, wherein the second complex, if present, provides a color signal.

9. The method of claim 8, further comprising quantifying the color signal.

10. The method of claim 6, further comprising determining the third complex comprised by the third zone.

11. The method of claim 10, wherein the third complex, if present, provides a color signal.

12. The method of claim 11, further comprising quantifying the color signal.

13. The method of claim 1, wherein the beta-1 transferrin comprised by the beta-1 transferrin-depleted sample is less than about 1% by weight of total protein comprised by the sample.

14-17. (canceled)

18. The method of claim 8, wherein the time interval between the subjecting the biological sample to the solid support and the providing a color signal is less than about 1 hr.

19-21. (canceled)

22. The method of claim 1, wherein the biological sample is selected from the group consisting of a serum sample, a plasma sample, a nasal fluid sample and a aural fluid sample.

23. The method of claim 1, wherein the sialic acid-specific lectin is selected from the group consisting of Allomyrina dichotoma agglutinin (allo A) and Sambucus nigra lectin (SNA-I).

24. The method of claim 1, wherein the volume of an undiluted biological sample is less than about 20 μl.

25-27. (canceled)

28. A system for performing a lateral flow immunoassay for determining presence, absence or quantity of cerebral spinal fluid in a beta-1 transferrin-depleted biological sample, the system comprising a separation section and a lateral flow immunoassay section, wherein the lateral flow immunoassay section comprises a substantially horizontally disposed test surface comprising a first zone comprising a first conjugate comprising a first antibody directed against transferrin and colloidal gold particles, and a second zone comprising a second antibody directed against transferrin, wherein the second antibody is immobilized on the test surface in the second zone.

29. The system of claim 28, wherein the test surface further comprises a third zone comprising a third antibody directed against the first antibody.

30. The system of claim 28, wherein the first antibody is a polyclonal antibody and the second antibody is a monoclonal antibody.

31. The system of claim 28, wherein the first antibody is a monoclonal antibody and the second antibody is a polyclonal antibody.

32-33. (canceled)
